(19) United States
(12) Patent Application Publication
(10) Pub. No.: US 2009/0110638 A1
(43) Pub. Date: Apr. 30, 2009

(54) METHODS USING THE GRUENEBERG GANGLION CHEMOSENSORY SYSTEM

(75) Inventor: David S. KOOS, Pasadena, CA (US)

Correspondence Address: MORGAN LEWIS & BOCKIUS LLP, 1111 PENNSYLVANIA AVENUE NW, WASHINGTON, DC, 20004, US

(73) Assignee: CALIFORNIA INSTITUTE OF TECHNOLOGY, Pasadena, CA (US)

(21) Appl. No.: 12/257,616

(22) Filed: Oct. 24, 2008

Related U.S. Application Data
(60) Provisional application No. 61/000,264, filed on Oct. 24, 2007.
(60) Provisional application No. 61/190,126, filed on Aug. 26, 2008.

(51) Int. Cl. A61K 38/00; A61K 49/00; C12Q 1/48; C12Q 1/44; C12Q 1/02

(52) U.S. Cl. 424/9.1; 514/2; 435/15; 435/19; 435/29

(57) ABSTRACT

The present invention relates to physical and pharmaceutical methods of increasing or decreasing the activity of the Grueneberg Ganglion (GG) in a subject. In one embodiment, the method comprising administering a compound to the GG, wherein the compound is an agonist or antagonist, respectively, for at least one guanylyl cyclase receptor or the receptor's downstream effectors. The present invention also relates to methods of screening candidate compounds for their ability to modulate the activity of the GG. The present invention also relates to methods and kits for positively identifying a GG neuron based upon the presence or absence of the pGC-G or pGC-A receptors.

### CROSS-REFERENCE TO RELATED APPLICATIONS

The application claims priority to U.S. Provisional Application No. 61/000,264, filed 24 Oct. 2007 and U.S. Provisional Application No. 61/190,126, filed 26 Aug. 2008, both of which are incorporated by reference in their entirety.

### STATEMENT REGARDING FEDERALLY SPONSORED RESEARCH OR DEVELOPMENT

Part of the work performed during development of this invention utilized U.S. Government funds through National Institutes of Health Grant Nos R01 HD037105 and R01 HD043897, T32 NS07251, U54 NS39405 and R01 EY018241. The U.S. Government has certain rights in this invention.

### BACKGROUND OF THE INVENTION

1. Field of the Invention

The present invention relates to physical and pharmaceutical methods of increasing or decreasing the activity of the Grueneberg Ganglion (GG) in a subject. In one embodiment, the method comprising administering a compound to the GG, wherein the compound is an agonist or antagonist, respectively, for at least one guanylyl cyclase receptor or the receptor's downstream effectors. The present invention also relates to methods of screening candidate compounds for their ability to modulate the activity of the GG. The present invention also relates to methods and kits for positively identifying a GG neuron based upon the presence or absence of the pGC-G or pGC-A receptors.

2. Background of the Invention

Olfaction sense plays a crucial role in the survival of many animals. As a means to monitor the chemical environment for a variety of molecules, olfaction can direct and influence a broad spectrum of behaviors and physiological responses that directly impact the quality of an organism's life. This primal sense assists in locating and evaluating food sources, detecting predators and it also can influence the expression of social and sexual behaviors.

Behaviorally relevant chemical signals, often referred to as pheromones, are found in all sorts of animal secretions, such as urine, feces, saliva and secretions from the skin and internal glands. These chemical signals communicate an enormous amount of information about the animal producing the odor such as its gender, identity and familiarity, sexual maturity, reproductive status, social status, and stress level. Furthermore, reception of these chemical signals can lead to physiological changes in the recipient such as modifying the onset of puberty or influencing the rate of sperm production. Therefore, as an essential form of animal communication, pheromone type chemical signals are involved in a broad spectrum of fundamental behaviors such as courtship, mating, aggression, parental protection of offspring, nursing, neonatal attachment and the formation of food preferences. In addition to pheromone type signals, other environmental signals, such as odorants, inhaled molecules and even indicators of the immediate environmental may also provide signals that are detected by an animals' olfactory pathway that can drive and influence behavior and physiological responses.

While the importance of olfaction sense in animal behavior is well accepted, determination of the actual olfactory subsystem that mediates these processes remains unclear. The mammalian olfactory system contains at least four specialized neural chemosensory subsystems: (1) the main olfactory system, (2) the vomeronasal or accessory olfactory system, (3) the GC-D system and (4) the Grueneberg Ganglion. Each of these systems express distinct types of receptors, are spatially segregated within the nasal cavity and project to different regions of the brain suggesting that they serve as distinct avenues for the transmission of different types or classes of chemosensory information to the brain.

Historically, the vomeronasal subsystem was attributed with the function of detecting and transmitting the reception of all biologically relevant chemical signals. However, recent evidence has called this all-encompassing proposal into question. While it seems clear that the vomeronasal subsystem can influence some behaviors, various ablation studies have indicated that many other olfactory-guided behaviors are much less reliant on the vomeronasal system. In fact some mammals, including human, are thought not to have a functional vomeronasal system.

The Grueneberg Ganglion is the most externally accessible of the known mammalian olfactory chemosensory subsystems. The unique vestibular location of this nerve, just inside of the nostrils, combined with its unusual neural connectivity make the Grueneberg Ganglion ideally suited as a means to detect chemical signals that influence important olfactory-guided behaviors and physiological responses.

The Grueneberg Ganglion is composed of bilateral clusters of neurons located in the dorsal medial nasal vestibule just inside of the nostrils and far forward of the other two olfactory subsystems. From this extreme rostral region, the Grueneberg Ganglion neuron clusters project fascicles of axons ipsilaterally through the nasal cavity, into the cranial vault and onto the first relay station of the olfactory system, the olfactory bulbs. From here, the axons of the GG neurons form synaptic connections with the second order neural circuits that transmit chemosensory information deep into the brain. This direct wiring to the olfactory bulb, combined with its expression of pan-olfactory markers and chemosensory signal transduction components, indicate that the Grueneberg Ganglion serves as an avenue for the transmission of sensory information to the brain.

Modulating the neural activity of the Grueneberg Ganglion, either by stimulation or by inhibition, will have therapeutic value for the treatment of variety of human medical conditions such as mood disorders, social anxiety, stress, panic, neuro-endocrine functioning, body temperature control, sexual motivation, eating disorders and blood pressure disorders.

In addition to human therapeutic and psychological value, manipulation of the Grueneberg Ganglion holds great potential for livestock management and animal husbandry. Manipulating the activity of the Grueneberg Ganglion in livestock, pets and research animals will allow the animal handlers to alter aggressive behaviors and increase breeding and reproductive success. In addition, reducing anxiety or stress levels and reducing aggression can increase the quality of life of these animals.

### SUMMARY OF THE INVENTION

The present invention relates to methods of increasing or decreasing the activity of the Grueneberg Ganglion (GG) in a subject, the method comprising administering a compound to the GG, wherein the compound is an agonist or antagonist, respectively, for at least one guanylyl cyclase receptor or the receptor's downstream effectors.

The present invention also relates to methods of modulating the activity of the GG or the GG neurons, with the methods comprising altering the activity of the GG neurons by means other than directly affecting the activity of a guanylyl cyclase receptor. For example, the GG neurons may be stimulated electrically or with transcranial magnetic stimulation (TMS), pulsed laser induced stimulation and even changes in temperature. Other means of modulating the GG neurons include but are not limited to ligating or cutting the axonal extensions of the GG neurons, killing the GG neurons and providing a shunt to the nasal passages of the animal to prevent access of the inhaled compounds to the GG neurons. Methods of destroying cells are well known in the art and include such techniques as cauterization, chemical ablation, genetic ablation, such as knockout models and hyper-excitatory lesioning.

The present invention also relates to methods of screening candidate compounds for their ability to modulate the activity of the GG, with the methods comprising administering the candidate compound to a particulate guanylyl cyclase A receptor (pGC-A), a particulate guanylyl cyclase G receptor (pGC-G) or both, and determining the activity of the pGC-A, the pGC-G or both in response to the compound. The determined activity in response to the candidate compound is indicative that the compound can potentially modulate the activity of the GG.

The present invention also relates to methods and kits for positively identifying a GG neuron based upon the presence or absence of the pGC-G or pGC-A receptors.

### BRIEF DESCRIPTION OF THE DRAWINGS

FIG. 1 depicts the location of the Grueneberg Ganglion (GG) in the nasal cavity.

FIG. 2 depicts expression of phosphodiesterase 2A and 4A in the main olfactory epithelium (MOE) and the Grueneberg Ganglion (GG).

FIG. 3 depicts the immunostaining of particulate guanylyl cyclase D receptor (pGC-D) in the main olfactory epithelium (MOE) and in the Grueneberg Ganglion (GG). The Figure shows that the GG does not express pGC-D.

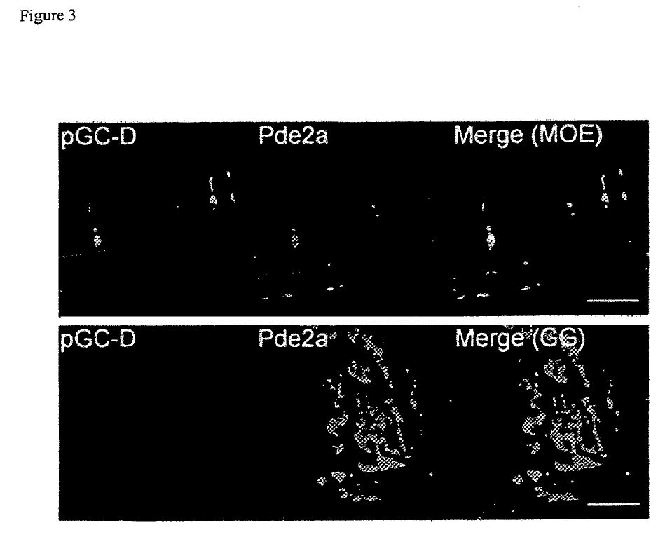

FIG. 4 depicts the expression of particulate guanylyl cyclase G receptor (pGC-G) and particulate guanylyl cyclase A receptor (pGC-A) in the Grueneberg Ganglion.

FIG. 5 depicts the expression of carbonic anhydrase II (CAII) in the Grueneberg Ganglion.

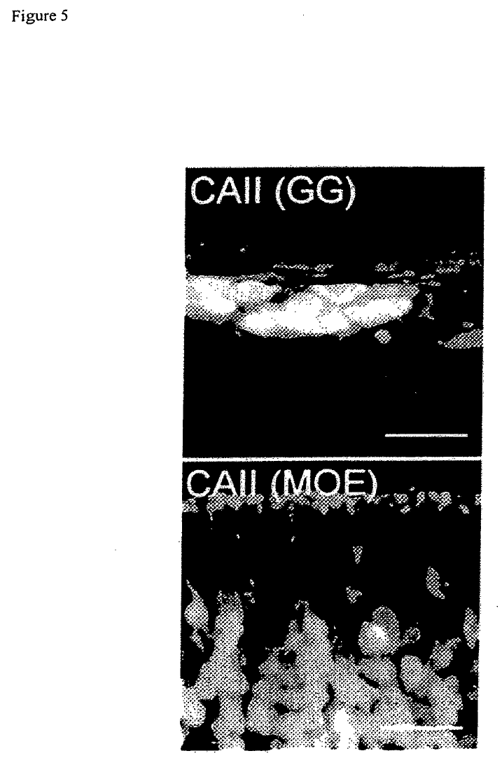

FIG. 6 depicts the signal transduction pathway of the pGC-A and pGC-G receptors on the extracellular surface of the neurons of the Grueneberg Ganglion.

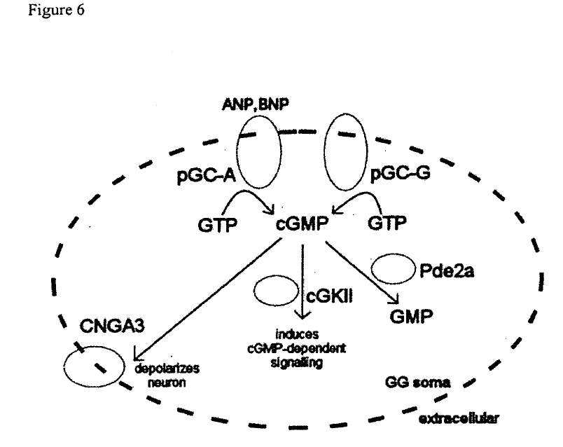

FIG. 7 depicts a TEM micrograph of a GG neuron showing clusters of ciliary basal bodies (1) supported by actin foundations (3) embedded deep within the soma near mitochondria (4) and nuclei (5). These cilia (2) run across the membrane of the cell in bundles and are held tight to the membrane by the glial-like ensheathing cells. Lower-right inset magnifies outlined area. Scale bar: 1 μm (inset: 0.3 μm).

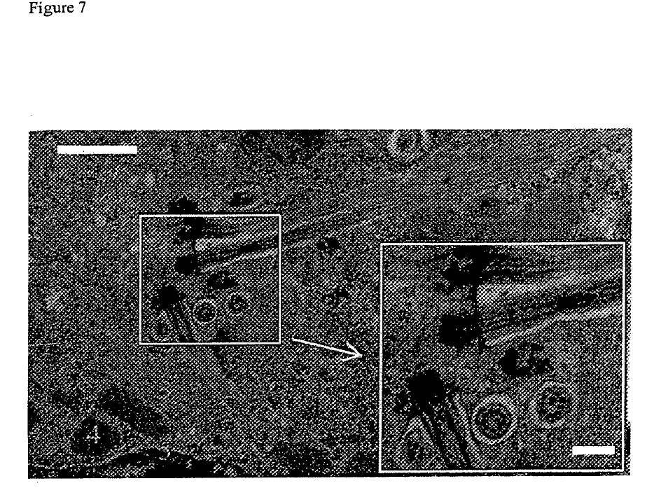

### DETAILED DESCRIPTION OF THE INVENTION

The present invention relates to methods of modulating the activity of Grueneberg Ganglion (GG) neurons in a subject. The GG is a compact cluster of neurons encased by glial cells located in the rostral portion of the nasal passages of mammals. See Brechbühl, J. et al., Science, 321(5892): 1092-1095 (2008), which is incorporated by reference. The GG neurons have, in general, round cell bodies with numerous primary cilia that project into, but not necessarily through, an outer layer of keratinized epithelium (KE). The outer KE layer, which is in direct contact with the airspace of the nostrils, apparently surrounds the GG neuronal cilia and this KE appears to be permeable to hydrophilic substances. Generally speaking, GG neurons express olfactory marker protein (OMP). Accordingly, GG neurons can be, at least in part, identified by their anatomical location and/or their expression of OMP. The methods of the current invention can be employed to verify the identity of the GG neurons.

As used herein, the term subject is used interchangeably with “patient” or “individual” and means a vertebrate with an olfactory system. In one embodiment, the animal is a mammal, such as, but not limited to, a rodent, canine, feline, bovine, equine, porcine and other livestock, deer, goats, sheep and human and non-human primates.

In one embodiment of the present invention, the activity GG neurons is inhibited when an antagonist is administered to the GG neuron. In another embodiment of the present invention, the activity GG neurons is increased when an agonist is administered to the GG neuron. As used herein, the “activity of a GG neuron” or “activity of GG neurons” is used to indicate any detectable signal that the neuron generates in response to a stimulus. In one embodiment, the activity is a signal that the neuron promulgates or could promulgate to another cell, such as a neighboring neuron. Detection of neuronal activity for the present invention can be accomplished by any means known in the art. One method of measuring or detecting the activity of a neuron includes detection or measurements of changes in action potentials, membrane potentials and receptor potentials compared to the resting membrane potential of a neuron. Other methods of detecting the activity of a neuron include measuring or detecting the propagation of an action potential from one neuron to the next, or measuring or detecting the voltage or ion influx/efflux along the neuron, or measuring or detecting the release of neurotransmitters into a synaptic cleft. Methods of determining or measuring activity of neurons are well known in the art and include, for example, patch clamps, monitoring levels of movement of labeled neurotransmitters, e.g., radioactively labeled neurotransmitters, MRI BOLD (blood oxygen level dependent) contrast, monitoring metabolism and metabolic breakdown products, monitoring glucose intake, monitoring Ca++ movement, monitoring expression of IEGs (immediate early genes) such as, but not limited to, cFos, Erg-1, ZENK and Arc. Methods of determining neuronal activity are disclosed in Principles of a Neural Science, 4^{th} Edition, Kandel, E. R., et al. (Eds). McGraw-Hill, 2000, and The Neuron: Cell and Molecular Biology, 3^{rd} Edition, Levitan, I. R., and Kaczmarek, L. K., Oxford University Press, New York, N.Y., 2001, both of which are incorporated by reference herein.

Additional methods of measuring the activity of neurons include measuring, detecting or monitoring behavioral responses in an animal. For example, Brechbühl et al. report that severance of the GG axonal projections abrogates the “freezing response” in mice that is normally associated with the detection of alarm pheromones. Thus, one method of determining the “activity of the GG” or the “activity of the GG neuron” in a subject would be to monitor the behavior of the animal in response to the compound. The behavior that is monitored in the animal should be well-correlated to the activity of the neurons or circuitry in question. The correlation between the behavior and a specific circuitry, however, need not be a standard or model that must be universally accepted in the scientific community for there to be a correlation between a behavior and the neuronal circuitry. Of course, a well-accepted behavioral model that correlates behavior with a specific circuitry could also be useful for evaluating neuronal activity in the methods of the present invention.

Additional methods of measuring or detecting the activity of a neuron include monitoring second messengers within the cells. Examples of second messengers include, but are not limited to, calcium ions (both from external sources and internal stores), cyclic GMP (cGMP) production or breakdown, cyclic AMP (cAMP) production or breakdown, generation or breakdown of inositol triphosphate (IP₃), generation or breakdown of diacyglycerides (DAGs), and even gene transcription in response to activation or inhibition of transcription factors. Numerous methods in the art exist for the detection and measurement of second messengers that can be used as direct or indirect measurements of the neuron activity.

For example, in one embodiment, changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11: 159-164 (1994) may be used to determine the level of cGMP. Further, an assay kit for measuring cAMP and/or cGMP is described in U.S. Pat. No. 4,115,538, herein incorporated by reference. Still another method for assessing intracellular cGMP is disclosed in U.S. Pat. No. 7,329,494, which is incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on signal transduction. A host cell containing a gene encoding a protein of interest, such as a marker protein, is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, green fluorescent protein, firefly luciferase, bacterial luciferase, β-galactosidase and alkaline phosphatase.

The amount of transcription is then compared to the amount of transcription in either the same cell or population of cells in the absence of the test compound, or it may be compared with the amount of transcription in a substantially identical cell or population of cells that lacks the protein of interest. A substantially identical cell or population of cells may be derived from the same cells from which the recombinant cell was prepared but which had not been modified by introduction of heterologous DNA. Any difference in the amount of transcription indicates that the test compound has, in some manner, altered the activity of the protein of interest.

“Inhibiting the activity” of the GG or a GG neuron means that the activity, however measured or determined, is less than the activity of an untreated GG or GG neuron. The inhibition need not be a total inhibition, provided there is a decrease in activity of the GG or GG neuron, compared to control levels. Moreover, the measured inhibition over controls need not be statistically significant, provided that the data exhibits at least a trend towards inhibition of the GG or GG neuron. “Increasing the activity” of the GG or a GG neuron means that the activity of the GG or the GG neuron is more than the activity of the untreated GG or GG neuron. The increase in activity can be at any level of activity of the GG or GG neuron, compared to control levels. Moreover, the measured increase over controls need not be statistically significant, provided that the data exhibits at least a trend towards increase of the GG or GG neuron. The control levels may be on the same subject or same group of cells prior to administration of the compound, with the activity of the treated GG or GG neuron being assessed on the same individual or same group of cells after treatment. Accordingly, the methods may further comprise measuring GG neuronal activity both before (as a control) and after treatment (as an experimental) on the identical subject, population of subjects, cell, group of cells or populations of cells. Of course, the methods may also comprise measuring or detecting activity in two separate individuals (or populations of individuals) or groups of cells (or populations of cells), with one population being the control, untreated group and the other population being the treated group.

Select embodiments of the methods of the present invention comprise administering a compound or composition to the GG or GG neurons. In one embodiment, the compound or composition is an antagonist that inhibits the activity of the GG or the GG neurons. In another embodiment, the compound or composition is an agonist that increases the activity of the GG or the GG neurons. The compounds or compositions can be administered to the GG or GG neurons in any manner designed to deliver the compound or composition to the GG or GG neurons. In one embodiment, the compounds or compositions are administered to the GG or GG neurons via inhalation. In another embodiment, the compounds or compositions are administered to the GG or GG neurons by topical application at the rostral portion of the inside the nostrils of the individual. Other routes of administration of the compounds or compositions of the present invention include but are not limited to oral, subcutaneous, intravenous, intraarterial, intraperitoneal and intramuscular to name a few.

The compounds or compositions of the present invention can be coadministered with other compounds of compositions. As used herein, the term “coadminister” is used to mean that each of at least two compounds are administered during a time frame wherein the respective periods of biological activity overlap. Thus the term includes sequential as well as coextensive administration of compounds or compositions. If more than one substance is coadministered, the routes of administration of the two or more substances need not be the same. The scope of the invention is not limited by the identity of the substance which may be coadministered. The compounds or compositions of the present invention may also be used in conjunction with other therapies, such as axonal lesioning or other treatments designed to physically or chemically alter the reactivity of the GG or the GG neurons.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions. In certain situations, delayed release preparations may be advantageous and compositions which can deliver an active ingredient in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdennal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986), which is incorporated by reference.

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration in the mouth include lozenges, pastilles and mouth washes.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants. They may also contain additional active agents in addition to the substance of the present invention.

The pharmaceutical compositions of the present invention include peptides, which may also be employed in accordance with the present invention by expression of polypeptides in vivo, often referred to as “gene therapy.”

Thus, for example, cells may be engineered with a polynucleotide (DNA or RNA) encoding for an active peptide ex vivo, the engineered cells are then provided to a patient to be treated with the polypeptide. Such methods are well-known in the art. For example, cells may be engineered by procedures known in the art by use of a retroviral particle containing RNA encoding the polypeptide of the present invention.

Similarly, cells may be engineered in vivo for expression of, for example, an active peptide in vivo, for example, by procedures known in the art. As known in the art, a producer cell for producing a retroviral particle containing RNA encoding a polypeptide of the present invention may be administered to a patient for engineering cells in vivo and expression of the polypeptide in vivo. These and other methods for administering a polypeptide of the present invention by such methods should be apparent to those skilled in the art from the teachings of the present invention. For example, the expression vehicle for engineering cells may be other than a retroviral particle, for example, an adenovirus, which may be used to engineer cells in vivo after combination with a suitable delivery vehicle.

Retroviruses from which retroviral plasmid vectors may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The producer cell line generates infectious retroviral vector particles which include the nucleic acid sequence(s) encoding the active peptides. Such retroviral vector particles then may be employed, to transduce eukaryotic cells, either in vitro or in vivo. The transduced eukaryotic cells will express the nucleic acid sequence(s) encoding the active peptide(s). Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Accordingly, the present invention also relates to methods of inhibiting or increasing the activity of the GG or GG neruons, with the methods comprising administering to the subject a pharmaceutically effective amount of an active peptide. As used herein, the term “active peptide” is a peptide, polypeptide or protein that affects the activity of the GG or the GG neruons.

The compounds or compositions of the present invention are designed to specifically interact, i.e., as an antagonist or agonist, with at least one guanylyl cyclase (GC) receptor. Guanylyl cyclase is, in general, found in a soluble form in the cytosol of cell and a membrane-bound particulate form. The methods of the present invention relate to particulate guanylyl cyclase (pGC). In particular, the compounds or compositions are designed to interact with the A and/or G isoforms of the GC receptors (GC-A and GC-G, respectively), and their functional orthologs in various species. Thus, the present invention relates to PGC-A and pGC-G in mice, as well as these same functional ortholog genes in humans, rats, dogs, livestock, etc, regardless of the name assigned to it in a particular species. The identity of these pGCs is readily identified based upon the amino acid and/or DNA sequences encoding the specific receptor. GC receptors, in general, comprise a single membrane-spanning domain of about 25 amino acids, a large N-terminal extracellular domain of about 500 amino acid residues or more, and a large C-terminal intracellular domain of about 500 amino acids. The intracellular domain, in turn, comprises a kinase-like domain (KLD), a catalytic domain (CD) and a region between the CD and KLD that is likely a dimerization domain (DD). The structure of the GC receptors, in particular the GC-A receptor, is described in Kuhn, M., Circ. Res., 93(8):700-709 (2003), which is incorporated by reference. The structure of the GC-G receptor is described in Schulz, S., et al. J. Biol. Chem., 273(2):1032-1037 (1998), which is incorporated by reference. In addition, the GenBank Accession numbers for pGC-A (Natriuretic peptide receptor 1) for mus musculus are NM_{—}008727 and NP_{—}032753, and for homo sapiens is NP_{—}000897. The GenBank records are incorporated by reference. The GenBank Accession numbers for pGC-G for mus musculus are NM_{—}001081076 and P_{—}001074545. The GenBank records are incorporated by reference.

Non-limiting examples of compounds that may interact with the pGCs of the present invention include both natural and synthetic peptides such as those peptides listed in U.S. Pat. Nos. 5,352,587, 6,407,211, 6,818,619, and 7,384,917, all of which are incorporated by reference. Other compounds that may interact with the pGCs of the present invention include, but are not limited to, those compounds disclosed in United States Pre-Grant Publication No 2006/0025367, which is incorporated by reference. Other compounds or compositions that may interact with the pGCs of the present invention include but are not limited to pheromones, odorants and inhaled molecules. Still other compounds that may interact with the pGCs of the present invention include but are not limited to c-type natriuretic peptides (CNPs) heat-stable enterotoxins, guanylin and uroguanylin. Still other factors that interact with the GG neurons include but are not limited to environmental factors such as temperature and atmospheric compositions.

Typically, the GC interacts with its ligand at the extracellular surface, which, in turn, causes the intracellular catalytic domain of the GC receptor to generate cGMP from GTP. The cGMP can then generate internal signaling via second messenger pathways. In general, peptides interact with GCs at the extracellular surface, but any molecule that causes GC activation or inactivation is useful in the present invention. For example, atrial natriuretic peptide (ANP) and B-type natriuretic peptide (BNP) are known agonists for the GC-A receptor. Other agonists or antagonists may be uncovered by employing the methods of the present invention. Additional antagonists would, of course, include antibodies that interfere with binding of the GC receptor and an agonist. Thus, as one of skill in the art would recognize, the term antagonist is used herein to mean a compound or composition that decreases the activity of the GC receptor. The mechanism of action of the antagonist can vary and can include typical mechanisms of action such as, but not limited to, competitive binding, counteracting the activity of the GG and decreasing the effectiveness of any second messenger or downstream effector systems or molecules. Likewise, the mechanism of action of an agonist can vary and can include typical mechanisms of action such as, but not limited to, increasing the activity of the GG and increasing the effectiveness or length of activity of any second messenger or downstream effector systems or molecules.

Second messengers of the GCs of the present invention may include, but are not limited to, calcium ions (both from external sources and internal stores), cyclic GMP (cGMP) production or breakdown, cyclic AMP (cAMP) production or breakdown, generation or breakdown of inositol triphosphate (IP₃), generation or breakdown of diacyglycerides (DAGs), and even gene transcription in response to activation or inhibition of transcription factors. For example, the inventors have discovered that GG neurons express cyclic nucleotide gated channels (CNGs) that can mediate depolarization in response to a buildup of internal cyclic nucleotides, such as cGMP. In general, CNGs conduct Na+ and/or Ca++ influx in response to increases in cyclic nucleotides, such as cGMP. In particular, GG neurons are presently shown to express the CNG alpha 3 (CNGA3) subunit, which heretofore was primarily associated with vision. Indeed, the only cells that were known to express CNGA3 subunits were cone cells of the retina and small subset of olfactory cells. See Biel, M. and Michalakis, S., Mol. Neurobiol., 35:266-277 (2007), which is incorporated by reference. The GenBank Accession numbers for cyclic nucleotide gated channel alpha 3 (CNGA3) for mus musculus are NM_{—}009918 are NP_{—}034048 and for homo sapiens is AAH96298. The GenBank records are incorporated by reference.

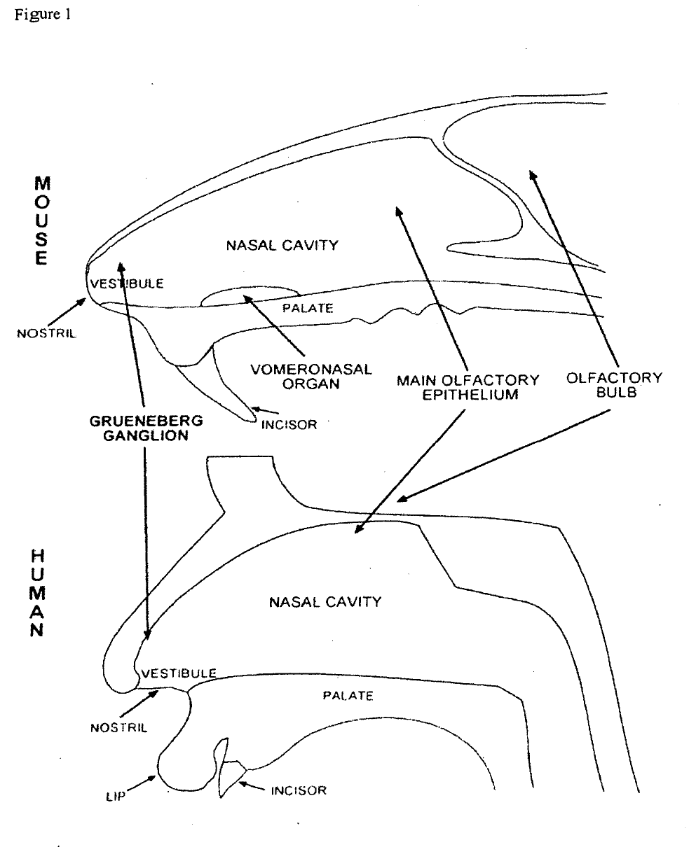

Accordingly, administering an antagonist or agonist of a “downstream effector” of the GC receptors of the present invention comprises the administration of compounds to GG neurons that interfere with or enhance ion influx through CNGs via the activation of at least one pGC. To be clear, for the purposes of the present invention, it is not enough to simply interfere with any second messenger, such as calcium, within the GG neurons. Rather, the methods comprise affecting a second messenger or downstream effector within the GC receptor, and this second messenger or downstream effector must be linked to the GC receptor itself. For example, the invention does not necessarily encompass depletion of Ca++ stores using a non-specific compound that depletes internal Ca++ stores, such as thapsigargin, since this intervention is not specifically linked to cGMP. FIG. 1 demonstrates the cGMP signal transduction pathway in the GG. For example, binding of atrial natriuretic peptide (ANP), B-type natriuretic peptide (BNP), or other ligands to pGC-A or pGC-G induces the conversion of GTP to cGMP by the particulate guanylyl cyclases. The produced cGMP can then open CNGA3 channels to depolarize the GG neuron, induce additional signaling through cGMP stimulated kinase II (cGKII), or be degraded to GMP by cGMP stimulated phosphodiesterase 2A. Accordingly, downstream effector molecules of cGMP generated from the pGCs of the present invention include, but are not limited to, cGMP stimulated kinase II (cGKII) and cGMP stimulated phosphodiesterase 2A (PDE2A). The Genbank accession number for the PDE2A protein for mus musculus, as of the date of this writing, is NP_{—}001008548 and for homo sapiens is NP_{—}002590. These GenBank records are incorporated by reference. PDE2A and methods of use are disclosed in United States Patent Pre-Grant Publication No. 2006/0168668, which is incorporated by reference. While this publication is directed to memory, one of skill in the art would readily adapt the methods disclosed therein for use in the present invention.

The present invention also relates to methods of screening candidate compounds for their ability to modulate the activity of the GG or GG neurons, with the methods comprising administering the candidate compound to a particulate guanylyl cyclase A receptor (pGC-A), a particulate guanylyl cyclase G receptor (pGC-G) or both, and determining the activity of the pGC-A, the pGC-G or both in response to the compound. The determined activity in response to the candidate compound is indicative that the compound can potentially modulate the activity of the GG.

The screening methods can be performed in vivo, on a subject, or can be performed in vitro on cells. In one embodiment, if the methods are performed in vivo, the methods may comprise administering a candidate compound directly to the GG by inhalation or topical application at the anatomically appropriate location. As used herein, a candidate compound is a compound or composition whose effects upon the GG are not known. A compound may be “suspected” of interacting with a pGC of the present invention, based upon preliminary data, and still be considered a “candidate compound” for the purposes of the present invention.

In one embodiment of the present invention, the methods are performed in vitro on host cells or groups of host cells that comprise a pGC-G and/or pGC-A of the present invention. As used herein, a “cell” can mean a single cell, or it can mean a population of cells. “Groups of cells” indicates more than more cell or more than one population of cells. A host cell in the present invention is a cell that expresses pGC-G and/or p-GC-A. The host cell can normally express the pGC-G and/or p-G C-A, or the host cell can be engineered to express the pGC-G and/or p-GC-A. The host cells that are engineered to express the pGC-G and/or p-GC-A can express a heterologous pGC-G and/or p-GC-A from a species different from the host cells, or the host cells that are engineered to express pGC-G and/or p-GC-A can express native pGC-G and/or p-GC-A that is normally expressed in same species as the host cells. For example, host cells can include mouse cells that are engineered to express or overexpress mouse pGC-G and/or p-GC-A, and host cells would also include human cells that are engineered to express mouse pGC-G and/or p-GC-A. Host cells also include GG cells, as well as skeletal muscle, lung and intestine cells that normally express pGC-G, and vascular smooth muscle, endothelium, nerve cells, adrenal cells, kidney, spleen and heart cells that normally express pGC-A.

Methods of engineering host cells are well known in the art. Accordingly, the present invention also relates to recombinant vectors, which include the isolated nucleic acid molecules of the present invention, and to host cells containing the recombinant vectors, as well as to methods of making such vectors and host cells and for using them.

Host cells are genetically engineered (transduced, transformed, or transfected) with the vectors of this invention which may be an expression vector. The vector may be, for example, in the form of a plasmid, a viral particle, a phage, etc. The engineered host cells can be cultured in conventional nutrient media modified as appropriate for activating promoters, selecting transformants, or amplifying the coding sequences of the pGC-G and/or p-GC-A of the invention. The culture conditions, such as temperature, pH and the like, are those previously used with the host cell selected for expression, and will be apparent to the skilled artisan.

The polynucleotides of the present invention may be employed for producing pGC-G and/or p-GC-A by recombinant techniques. Thus, for example, the polynucleotide sequence may be included in any one of a variety of expression vectors for expressing a polypeptide. Such vectors include chromosomal, nonchromosomal and synthetic DNA sequences, e.g., derivatives of SV40, bacterial plasmids, phage DNA, yeast plasmids, vectors derived from combinations of plasmids and phage DNA, viral DNA such as vaccinia, adenovirus, fowl pox virus, and pseudorabies. However, any other plasmid or vector may be used so long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli lac or trp, the phage lambda PL promoter and other promoters known to control expression of genes in prokaryotic or eukaryotic cells or their viruses. The expression vectors may also contain a ribosome binding site for translation initiation and a transcription terminator. The vector may also include appropriate sequences for amplifying expression.

In addition, the expression vectors may contain at least one selectable marker gene to provide a phenotypic trait for selection of transformed host cells. Such markers include dihydrofolate reductase (DHFR), GFP, or neomycin resistance for eukaryotic cell culture, and tetracycline or ampicillin resistance for culturing in E. coli and other bacteria.

The vector containing the appropriate DNA sequence as herein above described, as well as an appropriate promoter or control sequence, may be employed to transform an appropriate host to permit the host to express the pGC-G and/or p-GC-A. Representative examples of appropriate hosts, include but are not limited to: bacterial cells, such as E. coli, Salmonella typhimurium, fungal cells, such as yeast, insect cells, such as Drosophila S2 and Spodoptera Sf19, animal cells such as CHO, COS, and Bowes melanoma; and plant cells. The selection of an appropriate host is deemed to be within the scope of those skilled in the art from the teachings herein.

More particularly, the present invention also includes recombinant constructs comprising one or more of the sequences that encode pGC-G and/or p-GC-A. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example—bacterial: pQE70, pQE60 and pQE-9, available from Qiagen; pBS vectors, Phagescript vectors, Bluescript vectors, pNH88A, pNH16a, pNH18A, pNH46A, available from Stratagene; and ptrc99a, pKK223-3, pKK233-3, pDR540, pRIT5 available from Amersham. Among preferred eukaryotic vectors are pWLNEO, pSV2CAT, pOG44, pXT1 and pSG available from Stratagene; and pSVK3, pBPV, pMSG and pSVL available from Amersham. Other suitable vectors will be readily apparent to the skilled artisan.

In addition to the use of expression vectors in the practice of the present invention, the present invention further includes novel expression vectors comprising operator and promoter elements operatively linked to nucleotide sequences encoding a peptide of interest.

Among known bacterial promoters suitable for use in the production of proteins of the present invention include the E. coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter.

In a further embodiment, the present invention relates to host cells comprising vectors of the claimed invention. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be accomplished with calcium phosphate transfection, DEAE-Dextran mediated transfection, electroporation, transduction, infection, or other methods (Davis, L., et al., Basic Methods in Molecular Biology (1986), incorporated by reference).

The constructs in host cells can be used in a conventional manner to produce the gene product encoded by the recombinant sequence. Alternatively, the polypeptides of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, Cold Spring Harbor Laboratory, Cold Spring Harbor, N.Y. (1989), the disclosure of which is hereby incorporated by reference.

Transcription of a DNA encoding the polypeptides of the present invention by higher eukaryotes can be increased by inserting an enhancer sequence into the vector. Enhancers are cis-acting elements of DNA, usually about from 10 to 300 bp, that act on a promoter to increase its transcription efficiency or rate. Examples include the SV40 enhancer on the late side of the replication origin (bp 100 to 270), a cytomegalovirus early promoter enhancer, a polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter, The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Other vectors are commercially available.

Following transformation of a suitable host strain and growth of the host strain to an appropriate cell density, the selected promoter is derepressed by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, well known to those skilled in the art, including freeze-thaw cycling, sonication, mechanical disruption, or use of cell lysing agents.

Various mammalian cell culture systems can also be employed to express pGC-G and/or p-GC-A. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements.

A host strain may be chosen by its ability to modulate the expression of the inserted gene sequences, or to modify and process the gene product in the specific fashion desired. Expression from certain promoters can be elevated in the presence of certain inducers; thus expression of the genetically engineered pGC-G and/or p-GC-A may be controlled. Furthermore, different host cells have characteristics and specific mechanisms for the translational and post-translational processing and modification, e.g., glycosylation, phosphorylation, cleavage, of proteins. Appropriate cell lines can be chosen to ensure the desired modifications and processing of the protein expressed.

Additional post-translational modifications encompassed by the invention include, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein.

The present invention also provides methods of identifying a GG neuron, with the method comprising administering at least one labeling agent to a cell or group of cells that is suspected of being a GG neuron. As used herein a cell or group of cells is a “suspect” cell or group of cells based upon anatomical location of the neuron within an animal, or based upon the presence of other GG markers such as, but not limited to, OMP, CNGA3, PDE2A, cGKII etc. The presence of these additional GG markers need not be known prior to performing the methods the present invention and can optionally be detected at a later time.

Accordingly, as used herein, a “cell or group of cells suspected of being a GG neuron” is used to indicate that the technician may or may not have any prior knowledge or pre-conceived notion of the true identity of the cell or group of cells being tested. In one embodiment, it is known that the suspect cell or group of cells is positive for OMP, prior to administration of the labeling agent. In another embodiment, it is not known that the suspect cell or group of cells is positive for OMP prior to administration of the labeling agent. In this embodiment, the suspect cell or group of cells is labeled with two labeling agents, with the second labeling agent indicating the presence of OMP. The presence of both markers (OMP and pGC-A/p-GC-G) would indicate that the suspect cell or group of cells is a GG neuron.

The labeling agent that is applied to the cell or group of cells should specifically bind to particulate guanylyl cyclase A receptor (pGC-A) or particulate guanylyl cyclase G receptor (pGC-G) or both. Antibodies specific for pGC-A and pGC-G are known in the art and described below and also disclosed in the references which are listed below and incorporated by reference. Once labeled, the excess label is removed, if any excess is present, and the presence or absence of the label is determined. The presence of the labeling agent on the suspected cell or group of cells indicates that the cell is a GG neuron. Other examples of labeling agents can include vectors where, for example, a fluorescent protein is linked to the promoter for pGC-A or pGC-G. In this instance, the cells are transfected with the expression vector and the presence of the fluorescent protein, for example, GFP, would indicate that pGC-A or pGC-G is normally expressed in the suspect cell or group of cells. Thus, as used herein, “labeling a cell” includes transfecting a cell with an expression vector, where the expressed protein is a label that indicates that the cell may normally express pGC-G and/or pGC-A.

The present invention also relates to kits for performing the various methods of the present invention. The kits may include, but are not limited to, the labeling agents, e.g., labeled antibodies, that specifically label pGC-A and/or pGC-G, labeling agents that label a CNG, labeling agents that label OMP, cGMP, etc. The kits may also include reagents useful for cell culture and/or reagents useful for making a labeling solution, such as, but not limited to, buffers, antibiotics, vitamin solutions and the like. Other components may include expression vectors, primers, probes, cells, microwell plates and the like.

The present invention also relates to methods of modulating the activity of the GG or the GG neurons, with the methods comprising altering the activity of the GG neurons by means other than directly affecting the activity of a guanylyl cyclase receptor. As used herein, a means that affects a GG neuron “other than directly affecting the activity of a guanylyl cyclase receptor” indicates that the methods affect the neuron more generally, rather than a specific receptor or its downstream effector molecules. A method that affects the neuron on a more general level will likely affect one or more GC receptors and their downstream effector molecules. For example, the GG neurons may be stimulated electrically or with transcranial magnetic stimulation (TMS), pulsed laser, ultrasound stimulation. In these embodiments, an apparatus or stimulus is brought into physical contact with the GG neuron, but the apparatus or stimulus need not interact only with a GC receptor. Methods of stimulating a neuron with an electrically, magnetically and with lasers are well-known in the art. Other methods of modulating the GG neurons include, but are not limited to, changing the ambient temperature, ligating or cutting the axonal extensions of the GG neurons, killing the GG neurons and providing a shunt to the nasal passages of the animal to prevent access of the inhaled compounds to the GG neurons. Methods of destroying cells are well known in the art and include such techniques as cauterization, chemical ablation, genetic ablation, such as knockout models, and hyper-excitatory lesioning. Methods of modulating neurons, in general, can be found in the “Handbook of Experimental Neurology: Methods and Techniques in Animal Research” Tatlisumak, T. and Fisher, M. (Eds.), Cambridge University Press, New York, N.Y., (2006), which is incorporated by reference.

The following examples are meant to illustrate various embodiments of the present invention and are not intended to limit the scope of the subject matter described herein.

### EXAMPLES

### Example 1

### Methods and Materials

Mice were maintained as per IACUC-approved protocol. All animals used were postnatal. For immunohistochemistry, young and adult animals were euthanized with CO₂ and decapitated. Isolated nasal vestibules and other olfactory organs were immersion fixed in 4% paraformaldehyde/phosphate buffered saline (PBS) overnight at 4° C. From these preparations, either thin cryosections (18 μm), thick agarose-embedded sections (100 μm), or whole-mount nasal epithelia were extracted containing the GG organ.

Tissue was placed in blocking solution consisting of 0.3% Triton-X in PBS with 4% normal goat serum for at least 4 h at room temperature. Tissue was then incubated with primary antibody (with Triton-X and serum) overnight at 4° C. The next day, secondary antibody was washed off with at least 4 changes of PBS. Secondary antibody was then applied for 2 h at room temperature or overnight at 4° C. After washing, tertiary label (with Triton-X but no serum) was then applied to tissue for 2 h at room temperature. For antibody stainings with diaminobenzidine (DAB) chromogen development, tissue was washed with PBS and bleached with 0.3% hydrogen peroxide in PBS before the staining process was started.

For fluorescently-labeled tissue, final PBS washes included 0.2 μM Topro-3 (Invitrogen) as a nuclear stain. Tissue was mounted with Gel-Mount (Biomeda) or PBS and coverslipped. For DAB-developed tissue, final washes were performed in Tris-buffered saline. Signal was developed with a Ni-enhanced DAB kit (Thermo Scientific) with a development time of 5-12 minutes in the dark. DAB-developed tissue was then washed in multiple changes of PBS. Tissue was mounted in 100% glycerol, or dehydrated in ascending ethanol/water series, cleared in xylenes, mounted in Krystalon (EMD Chemicals), and coverslipped.

Polyclonal primary antibodies that were used are described in Table 1, below. Secondary antibody was biotinylated anti-rabbit IgG with normal goat serum, raised in goat (Vector Labs) at 3 μg/mL working concentration for thin sections or 0.75 μg/mL working concentration for thick sections or whole-mounts. For fluorescent labeling, tertiary label was streptavidin-conjugated Alexa 555 dye (Invitrogen) at 2 μg/mL working concentration. For DAB-labeling, tertiary label was streptavidin-conjulated horseradish peroxidase (MP Biomedical) at 1:500 working dilution.

TABLE 1
Antibodies Used

| Antigen | Source | Company | Working Dilution |
|---|---|---|---|
| Pde2a | Rabbit | Fabgennix (#PD2A-101AP) | 1:500-1:2000 |
| Pde4a | Rabbit | Fabgennix (#PD4-112AP) | 1:250 |
| pGC-A | Rabbit | Fabgennix (#PGCA-101AP) | 1:200-1:500 |
| pGC-B | Rabbit | Fabgennix (#PGCB-201AP) | 1:200 |
| pGC-C | Rabbit | Fabgennix (#PGCC-301AP) | 1:200 |
| pGC-E | Rabbit | Fabgennix (#PGCE-501AP) | 1:500-1:1250 |
| pGC-F | Rabbit | Fabgennix (#PGCF-601AP) | 1:200-1:800 |
| pGC-G | Rabbit | Fabgennix (#PGCG-701AP) | 1:100-1:500 |
| cGKII | Rabbit | Abgent (#AP8001a) | 1:50 |
| CNGA3 | Rabbit | Provided by X.-Q. Ding | 1:1000 |

Mice were anesthetized with Avertin (2-2-2-tribromoethanol, Sigma) and transcardially perfused with fixative (4% paraformaldehyde, 1.25% glutaraldehyde (Ted Pella), in 0.06M phosphate buffer, pH 7.4, 37° C.). Isolated nares were trimmed of excess tissue and post-fixed (4% paraformaldehyde, 1.25% glutaraldehyde, in 0.1 M NaCacodylate, pH 7.4, 4° C.). Tissue was then stained for about 1 h at 4° C. in 2% OsO4 in veronal acetate +5% sucrose, washed with 0.1 M maleate buffer (pH 5), and then incubated for about 1 h at room temperature with 1% uranyl acetate in maleate buffer. After dehydration through a graded ethanol series, tissue was embedded in ascending propylene oxide/Epon resin (Fluka) solutions until 100% Epon resin and solidified at 60° C. 120 nm semi-serial sections were cut and floated onto formvar-coated copper EM grids (Ted Pella) and then allowed to dry. The grids were then stained with uranyl acetate and lead citrate, rinsed with water, and then allowed to dry completely prior to EM capture.

For imaging, the samples were imaged on various Zeiss microscopes depending on the type of staining and the required resolution. For confocal imaging, high-magnification (40×-63X>1.2NA oil objectives) fluorescent images were generated from z-projections of approximately 1 μm-thick optical sections; lower-magnification (20× objective) fluorescent images were compiled from approximately 3 μm-thick optical sections.

Uniform adjustments to image brightness and contrast and noise reduction with a median filter were performed with ImageJ (NIH) and Paint Shop Pro (Corel). When comparisons were made between the GG and the MOE, the channel of comparison was imaged with the same power and gain, and subsequent brightness and contrast enhancements were made to the same level in both organs.

### Example 2

### Results

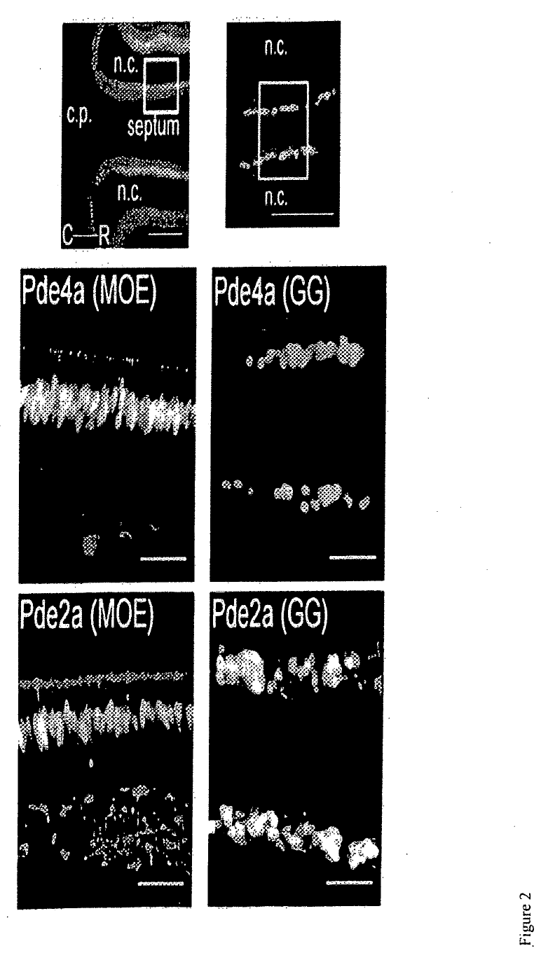

Phosphodiesterases degrade cyclic nucleotides that are essential second messengers involved in neuronal activation in most olfactory subsystems. The expression profile in the GG of two diagnostic phosphodiesterase isoforms implicates cGMP as the predominant second messenger molecule in this olfactory subsystem, similar to the neurons of the GC-D olfactory subsystem. The neurons of the GG express the cGMP-stimulated phosphodiesterase 2a (PDE2A) but not the cAMP-specific phosphodiesterase 4a (PDE4A) found in the olfactory sensory neurons (OSNs) of the main olfactory system (MOS). To facilitate visualization of the GG, a strain of mice (OMP-GFP) was used in which all of the primary olfactory sensory neurons of all olfactory subsystems are highlighted by the expression of GFP. See Ring G., et al. J Comp Neurol. 388(3):415-434 (1997), which is incorporated by reference. In neonatal mice, an antibody to the cAMP-selective PDE4A specifically labeled the highly olfactory marker protein (OMP)-expressing neurons in the main olfactory epithelium (MOE) (FIG. 2B). In contrast, OMP-expressing GG neurons in the rostral nasal vestibule were negative for PDE4A (FIG. 2A), suggesting that the cAMP signaling pathway commonly used in OSNs was not significantly employed in the GG neurons. In the MOE, the PDE2A antibody labeled only a small subset of neurons that expressed low levels of OMP in the caudal neuroepitheliary layers (FIG. 2C), corresponding to the GC-D neurons. In the nasal vestibule, strongly OMP-expressing GG neurons were intensely positive for PDE2A from birth to adult-aged mice.

Downstream signaling effects of cGMP generation are mediated by cGMP-dependent kinases. These cGMP-dependent kinases are known to exist in three isoforms, the splice-variants cGKIα and cGKIβ, and cGKII. In adult OMP-GFP mice, a cGKII antibody was able to bind to OMP-expressing GG neurons in the nasal vestibule, suggesting that cGMP produced by guanylyl cyclases has downstream signaling effectors in the GG olfactory subsystem.

To further investigate the molecular nature of the GG neurons, the expression of cyclic nucleotide gated (CNG) channels was investigated. In the neurons of the MOS, the tetrameric CNG channel composed of two CNGA2, one CNGA4, and one CNGB1b subunits depolarizes the neuron during elevation of cGMP or cAMP levels. These channel subunits, however, are not expressed in the GG. The CNGA3 subunit, which exhibits a strong selectivity for cGMP over cAMP, is expressed on the ciliary receptive domains of GC-D neurons, where it contributes to their chemosensory function by mediating calcium bursts in response to pGC-D-mediated cGMP production.

GG neurons were shown to express the CNGA3 subunit, similar to the GC-D olfactory subsystem and to cone cells of the retina. A previously-validated antibody specific to mouse CNGA3 (See Matveev A. V., et al., J Neurochem. 106:2042-2055 (2008) which is incorporated by reference) labeled morphologically-unusual subcellular domains on the somata representing the ciliary receptive structures of OMP-expressing GG neurons. The receptive cilia of the GC-D neurons are also decorated with CNGA3.

In the GC-D olfactory subsystem, a particulate guanylyl cyclase isoform (pGC-D) produces cGMP in response to binding of guanylin and uroguanylin peptide urinary derivates and subsequently triggers neuronal activation. In this manner, pGC-D contributes to the chemosensory capabilities of the GC-D neurons.

Surprisingly, however, it was determined that the GG does not express the pGC-D receptor protein, in contrast to the neurons of the GC-D olfactory subsystem. Thus, even though the GG employs a similar cGMP signal transduction pathway as the GC-D neurons, the receptor repertoires of these two olfactory subsystems are not equivalent.

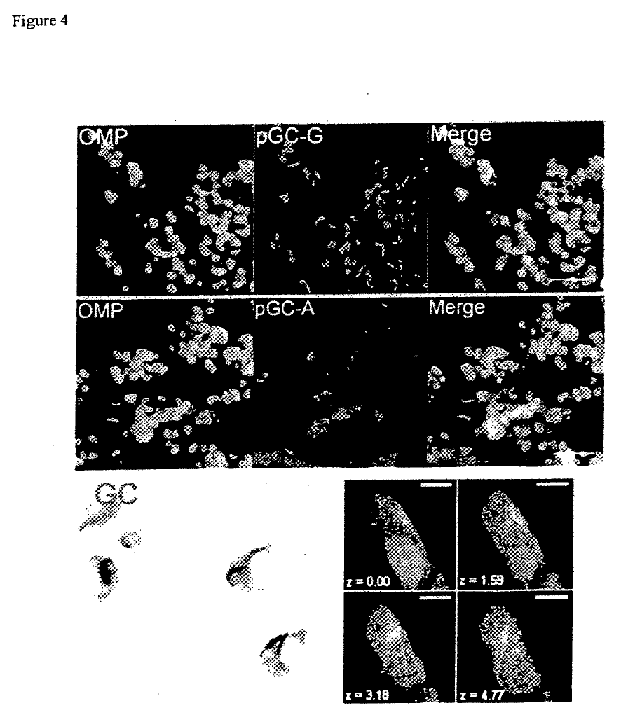

Only antisera directed against two isofomms, pGC-A and pGC-G, were able to distinctly label neurons of the GG organ in the nasal vestibule. Neither of these pGC isoforms has been previously detected in any of the olfactory subsystems in the primary olfactory pathway. The expression of pGC-G was detected on most of the GG neurons and was found in both neonatal and adult ages (FIG. 4A). pGC-G was not found on GC-D neurons. In contrast to the ganglion wide expression of pGC-G, expression of pGC-A was detected in only a small subset of GG neurons that were broadly distributed throughout the GG organ (FIG. 4B).

Moreover, pGC-G expression was restricted to subcellular domains on the somata representing the ciliary receptive structures of GG neurons, which was very similar to the CNGA3 staining. In thin sections of the OMP-GFP nasal vestibule imaged at high magnification, the pGC-G positive region appeared to be localized to thin fibers on the peripheries of the GG soma, with no observable directional preference. These fibers mostly appeared to follow the contours of GG cells in a “whip-like” fashion (FIGS. 4A and B). In some cases, the fibers extended from one GG neuron to touch a neighboring GG neuron. However, it did not appear that any of these whip-like fibers protruded from a GG neuron into the nasal cavity.

Despite being localized to the periphery of the GG neurons, pGC-G protein is not expressed by the tight-fitting glial cells that ensheath the GG neurons. To visualize the ensheathing glia of the nasal vestibule, the PLP-GFP mouse line was used. See Mallon B. S., et al., J Neurosci, 22(3):876-885 (2002), which is incorporated by reference. The PLP-GFP mouse line utilizes the proteolipid protein (PLP) promoter to drive expression of GFP. In this mouse line, GFP-positive glial cells and their processes can be seen wrapping clusters of GG neurons. The pGC-G positive structures in the GG, however, did not co-localize with the GFP-positive fibers of these glial cells. Instead, the glial cells appeared to be encasing the pGC-G positive cells.

To identify the GG subcellular domains that express pGC-G, transmission electron microscopy was performed. In semi-serial thin sections of the adult nasal vestibule, it was shown that each GG neuron was decorated by a plurum of cilia whose ultrastructural morphologies were consistent with the pGC-G-labeled subcellular domains seen with light microscopy. These cilia are seen erupting out from clusters of centrioles deep within the cell soma. The cilia are grouped and appear as long bundles that follow the contour of the cell in a variety of orientations. The encasement of GG neurons by the ensheathing cells was tight enough that the ciliary bundles were observed to be partially embedded into the membrane of a GG cell. The localization of pGC-G to GG neuronal cilia suggests a critical contribution of pGC-G to chemosensory function of the GG olfactory subsystem.

### Example 3

### Methods of identifying Ligands for pGCs

If a pGC or CNG of interest is not already present in a host cell, e.g. HEK293 cells, the host cells can be engineered to comprise a pGC of interest or a CNG of interest using standard transfection techniques (Ausuebl et al., Current Protocols in Molecular Biology, (2001) John Wiley & Sons). Two days after transfection, approximately 50,000 cells/well for a 96-well plate and 10,000 cells/well for a 384-well plate may be used to create a confluent cell monolayer with a plating volume of about 100 μL/well for 96-well plates or 25 μL/well for 384-well plates.

Cell plates may be removed from the incubator after overnight incubation. An equal volume of Loading Buffer with a membrane potential dye (Molecular Devices, Sunnyvale, Calif.) can be added to each well (100 μL per well for 96-well plates, 25 μL for 384-well plates) and the cell plates further incubated for 30 minutes at 37° C. After incubation, the plates can be directly assayed using a FLIPR (Fluorometric Imaging Plate Reader, Molecular Devices, Sunnyvale, Calif.) or FlexStation (Molecular Devices, Sunnyvale, Calif.) analysis.

Candidate natural synthetic ligand collections can be obtained and diluted to concentrations ranging from 1 nM to 10 μM for testing. Membrane potential assays will be performed immediately following the addition of the compounds as described in the FLIPR system manual for membrane potential assay. Such assays can also be performed in the presence of Ca++ sensitive dyes.

### Example 4

### Methods of Identifying Compounds or Compositions that Regulate The Activity of cGMP or its Downstream Effectors

Host cells are transfected, if necessary, with an expression vector encoding a pGC-G or pGC-A receptor of the present invention according to established methods in the art. To evaluate if compounds or compositions can act as agonists or antagonists to the pGC-G or P-GC-A receptors, a cGMP assay (CisBio International, Bedford, Mass., U.S.A.) can be run on the host cells. The product insert for the CisBio International cGMP assay is incorporated by reference. Other methods exist in the art for assessing cGMP levels and the invention is not limited to the method of detecting cGMP levels or methods of detecting levels of its downstream effectors.

For example, a control population of host cells can be maintained under normal culture conditions appropriate for the host cell type. A second population of host cells (experimental population) can be cultured under identical conditions, except that experimental population of cells is incubated with a candidate compound or composition, e.g., a peptide. Levels of cGMP are then assessed in both the control and experimental populations.

Host cells that demonstrate an increase in cGMP in response to the candidate compound can then be investigated further as an agonist of pGC-G or pGC-A, depending on the type of receptor present. Similarly, Host cells that demonstrate a decrease in cGMP in response to the candidate compound can then be investigated further as an antagonist of pGC-G or pGC-A, depending on the type of receptor present.

Once, for example, an agonist is identified with the protocols described herein, the agonist can be mixed with an additional experimental compound or composition to test for substances that affect the downstream effector molecules. A previously identified agonist can be incubated with, for example, erythro-9-(2-hydroxy-3-nonyl)adenine (EHNA) which is a known inhibitor of PDE2A, or with isobutylmethylxanthine (IBMX), which is a non-specific inhibitor of PDEs. The addition of a PDE2A inhibitor, whether a specific or non-specific inhibitor, can potentially prolong the effects of the receptor antagonist by preventing cGMP degradation. The levels of cGMP can in cells incubated with an agonist and a PDE2A inhibitor be assessed according to the methods described herein.

### Example 5

### Electrical Stimulation of the GG

A small electronically coupled stimulating nasal insert or stent is inserted into one or both of the patient's or animals nostrils. Small electric currents generated from a battery (or other electric source) are then delivered through the nasal insert's conductive pad directly onto the receptive fields of the Grueneberg ganglion nerve cells. These small electric shocks would lead to stimulation of the Grueneberg ganglion nerves and generate neural impulses that are then transmitted to the appropriate regions of the brain to elicit the desired behaviors and/or physiology changes.

The stimulating nasal insert must be able to stay in nostril and nasal vestibule region without the need of additional clips. To accomplish this, the stimulating nasal insert will be made from deformable material that is slightly larger in diameter that the recipient's nasal vestibule and be shaped with outer surfaces that correspond generally to the surfaces that are characteristic of the nasal vestibule of the human or animal to be treated.

The stimulating nasal insert should not completely block the nasal opening and therefore allow nose breathing. To accommodate nose breathing an air passageway will be incorporated in the design. The outer surface of the entire nasal insert will be non-adherent to allow for easy removal of the nasal insert and subsequent reapplication.

On the surface of the inert is a small conductive pad that will be used to stimulate Grueneberg Ganglion. This conductive pad will be located on the surface of the insert where the insert comes in contact with the Grueneberg ganglion when inserted into the nasal vestibule. The conductive pad will deliver the electric pulses to the Grueneberg Ganglion. The conductive pad itself and its leads may be made from non-magnetic material (such as carbon fiber or copper) to allow use of the stimulating nasal insert in MRI studies.

This Grueneberg Ganglion manipulation overcomes the need to isolate the behaviorally relevant chemical signals. It is more amenable to situations where the wearing of odorous secretions would be unacceptable.

### Example 6

### Ablation of the GG Neurons

The Grueneberg Ganglion or its axonal projections are destroyed to prevent transmission of Grueneberg Ganglion to the glomeruli. These lesions are performed surgically, chemically or with photo-ablation technologies.

By destroying the Grueneberg ganglion or blocking its transmission capabilities suppression of specific behaviors and/or physiology changes that require Grueneberg Ganglion stimulation and activity can be achieved.

The following references are incorporated herein by reference.

### REFERENCES

Breer H, Fleischer J, & Strotmann J (2006) The sense of smell: multiple olfactory subsystems. Cell Mol Life Sci 63(13):1465-1475.
Firestein S (2001) How the olfactory system makes sense of scents. Nature 413(6852):211-218.
Stowers L, Holy T E, Meister M, Dulac C, & Koentges G (2002) Loss of sex discrimination and male-male aggression in mice deficient for TRP2. Science 295(5559): 1493-1500.
Halpern M (1987) The organization and function of the vomeronasal system. Annu Rev Neurosci 10:325-362.
Brechbuhl J, Klaey M, & Broillet M C (2008) Grueneberg ganglion cells mediate alarm pheromone detection in mice. Science 321(5892):1092-1095.
Gruneberg H (1973) A ganglion probably belonging to the N. terminalis system in the nasal mucosa of the mouse. Z Anat Entwicklungsgesch 140(1):39-52.
Fleischer J, Hass N, Schwarzenbacher K, Besser S, & Breer H (2006) A novel population of neuronal cells expressing the olfactory marker protein (OMP) in the anterior/dorsal region of the nasal cavity. Histochem Cell Biol 125(4):337-349.
Fuss S H, Omura M, & Mombaerts P (2005) The Grueneberg ganglion of the mouse projects axons to glomeruli in the olfactory bulb. Eur J Neurosci 22(10):2649-2654.
Koos D S & Fraser S E (2005) The Grueneberg ganglion projects to the olfactory bulb. Neuroreport 16(17):1929-1932.
Roppolo D, Ribaud V, Jungo V P, Luscher C, & Rodriguez I (2006) Projection of the Gruneberg ganglion to the mouse olfactory bulb. Eur J Neurosci 23(11):2887-2894.
Storan M J & Key B (2006) Septal organ of Gruneberg is part of the olfactory system. J Comp Neurol 494(5):834-844.
Shinoda K, Ohtsuki T, Nagano M, & Okumura T (1993) A possible functional necklace formed by placental antigen X-P2-immunoreactive and intensely acetylcholinesterase-reactive (PAX/IAE) glomerular complexes in the rat olfactory bulb. Brain Res 618(1):160-166.
Shinoda K, Shiotani Y, & Osawa Y (1989) “Necklace olfactory glomeruli” form unique components of the rat primary olfactory system. J Comp Neurol 284(3):362-373.
Fleischer J, Schwarzenbacher K, Besser S, Hass N, & Breer H (2006) Olfactory receptors and signalling elements in the Grueneberg ganglion. J Neurochem 98(2):543-554.
Fleischer J, Schwarzenbacher K, & Breer H (2007) Expression of trace amine-associated receptors in the Grueneberg ganglion. Chem Senses 32(6):623-631.
Fulle H J, et al., (1995) A receptor guanylyl cyclase expressed specifically in olfactory sensory neurons. Proc Natl Acad Sci USA 92(8):3571-3575.
Juilfs D M, et al., (1997) A subset of olfactory neurons that selectively express cGMP-stimulated phosphodiesterase (PDE2) and guanylyl cyclase-D define a unique olfactory signal transduction pathway. Proc Natl Acad Sci USA 94(7):3388-3395.
Meyer M R, Angele A, Kremmer E, Kaupp U B, & Muller F (2000) A cGMP-signaling pathway in a subset of olfactory sensory neurons. Proc Natl Acad Sci USA 97(19):10595-10600.
Bender A T & Beavo J A (2006) Cyclic nucleotide phosphodiesterases: molecular regulation to clinical use. Pharmacol Rev 58(3):488-520.
Potter S M, et al., (2001) Structure and emergence of specific olfactory glomeruli in the mouse. J Neurosci 21(24):9713-9723.
Ring G, Mezza R C, & Schwob J E (1997) Immunohistochemical identification of discrete subsets of rat olfactory neurons and the glomeruli that they innervate. J Comp Neurol 388(3):415-434.
Feil R, Lohmann S M, de Jonge H, Walter U, & Hofman F (2003) Cyclic GMP-dependent protein kinases and the cardiovascular system: insights from genetically modified mice. Circ Res 93(10):907-916.
Biel M & Michalakis S (2007) Function and dysfunction of CNG channels: insights from channelopathies and mouse models. Mol Neurobiol 35(3):266-277.
Tanaka J C, Eccleston J F, & Furman R E (1989) Photoreceptor channel activation by nucleotide derivatives. Biochemistry 28(7):2776-2784.
Leinders-Zufall T, et al., (2007) Contribution of the receptor guanylyl cyclase GC-D to chemosensory function in the olfactory epithelium. Proc Natl Acad Sci USA 104(36):14507-14512.
Matveev A V, Quiambao A B, Browning Fitzgerald J, & Ding X Q (2008) Native cone photoreceptor cyclic nucleotide-gated channel is a heterotetrameric complex comprising both CNGA3 and CNGB3: a study using the cone-dominant retina of Nrl−/− mice. J Neurochem. 106:2042-2055.
Hu J, et al. (2007) Detection of near-atmospheric concentrations of CO2 by an olfactory subsystem in the mouse. Science 317(5840):953-957.
Walz A, Feinstein P, Khan M, & Mombaerts P (2007) Axonal wiring of guanylate cyclase-D-expressing olfactory neurons is dependent on neuropilin 2 and semaphorin 3F. Development 134(22):4063-4072.
Kuhn M (2003) Structure, regulation, and function of mammalian membrane guanylyl cyclase receptors, with a focus on guanylyl cyclase-A. Circ Res 93(8):700-709.
Kuhn M, et al. (2004) Identification of an orphan guanylate cyclase receptor selectively expressed in mouse testis. Biochem J 379 (Pt 2):385-393.
Schulz S, Wedel B J, Matthews A, & Garbers D L (1998) The cloning and expression of a new guanylyl cyclase orphan receptor. J Biol Chem 273(2):1032-1037.
Mallon B S, Shick H E, Kidd G J, & Macklin W B (2002) Proteolipid promoter activity distinguishes two populations of NG2-positive cells throughout neonatal cortical development. J Neurosci 22(3):876-885.
Tachibana T, Fujiwara N, & Nawa T (1990) The ultrastructure of the ganglionated nerve plexus in the nasal vestibular mucosa of the musk shrew (Suncus murinus, insectivora). Arch Histol Cytol 53(2):147-156.
Baker H, et al. (1999) Targeted deletion of a cyclic nucleotide-gated channel subunit (OCNC1): biochemical and morphological consequences in adult mice. J Neurosci 19(21):9313-9321.
Trinh K & Storm D R (2003) Vomeronasal organ detects odorants in absence of signaling through main olfactory epithelium. Nat Neurosci 6(5):519-525.

What is claimed is:

1. A method of inhibiting the activity of a Grueneberg Ganglion (GG) neuron in a subject, the method comprising administering a compound to the GG, wherein the compound is an antagonist for at least one guanylyl cyclase receptor or the receptor's downstream effectors, the antagonist being capable of inhibiting the activity of the GG.

2. The method of claim 1, wherein the at least one guanylyl cyclase receptor is a particulate guanylyl cyclase (pGC).

3. The method of claim 2, wherein the pGC is the PGC-A isoform, the pGC-G isoform or both.

4. The method of claim 3, wherein the pGC is the pGC-G isoform.

5. The method of claim 3, wherein the administration of the antagonist comprises inhalation of the antagonist, or topical application of the antagonist to rostral tip of the nostrils of the individual.

6. The method of claim 3, wherein the receptor's downstream effectors are selected from the group consisting of cGMP Kinase II, phosphodiesterase 2A (pde2a) and cyclic nucleotide gated channel 3 (CNGA3).

7. A method of increasing the activity of the Grueneberg Ganglion (GG) neuron in a subject, the method comprising administering a compound to the GG, wherein the compound is an agonist for at least one guanylyl cyclase receptor or the receptor's downstream effectors, the agonist being capable of increasing the activity of the GG.

8. The method of claim 7, wherein the at least one guanylyl cyclase receptor is a particulate guanylyl cyclase (pGC).

9. The method of claim 8, wherein the pGC is the pGC-A isoform, the pGC-G isoform or both.

10. The method of claim 9, wherein the pGC is the pGC-G isoform.

11. The method of claim 8, wherein the administration of the antagonist comprises inhalation of the antagonist, or topical application of the antagonist to rostral tip of the nostrils of the individual.

12. The method of claim 8, wherein the receptor's downstream effectors are selected from the group consisting of cGMP Kinase II, phosphodiesterase 2A (pde2a) and cyclic nucleotide gated channel 3 (CNGA3).

13. A method of screening a compound for its ability to modulate the activity of a Grueneberg Ganglion (GG) neuron, the method comprising
a) administering a compound to a particulate guanylyl cyclase A receptor (pGC-A), a particulate guanylyl cyclase G receptor (pGC-G) or both, and
b) determining the activity of the pGC-A, the pGC-G or both in response to the compound,
wherein the determined activity in response to the compound indicates that the compound can potentially modulate the activity of the GG.

14. The method of claim 13, wherein the methods are performed in vivo on a subject.

15. The method of claim 14, wherein the administration of the compound comprises inhalation of the compound, or topical application of the compound to rostral tip of the nostrils of the subject.

16. The method of claim 13, wherein the methods are performed on host cells in vitro, wherein the host cells comprise the pGC-A, the pGC-G or both.

17. The method of claim 16, wherein determining the activity of the pGC-A, the pGC-G or both comprises determining the levels of cyclic guanosine monophosphate (cGMP) responsive effectors.

18. The method of claim 17, wherein the cGMP responsive effectors are selected from the group consisting of cGMP Kinase II, phosphodiesterase 2A (pde2a) and cyclic nucleotide gated channel 3 (CNGA3).

19. A method of identifying a Grueneberg Ganglion (GG) neuron, the method comprising
a) administering at least one labeling agent to a cell or group of cells that is suspected of being a GG neuron, wherein the labeling agent indicates the presence of a particulate guanylyl cyclase A receptor (pGC-A) or the presence of a particulate guanylyl cyclase G receptor (pGC-G),
b) removing the excess of the at least one labeling agent, if any excess is present, and
c) determining which cell or group of cells is labeled with the at least one labeling agent,
wherein the presence of the at least one labeling agent on the suspected cell or group of cells indicates that the cell is a GG neuron.

20. The method of claim 19, wherein it is known that the cell or group of cells suspected of being a GG neuron is positive for the olfactory marker protein (OMP), prior to administration of the labeling agent.

21. The method of claim 19, wherein it is not known that the cell or group of cells suspected of being a GG neuron is positive for the olfactory marker protein (OMP), prior to administration of the labeling agent.

22. The method of claim 21, wherein the cell or group of cells is suspected of being a GG neuron based upon their anatomical location.

23. The method of claim 22, wherein the cell or group of cells suspected of being a GG neuron is labeled with two labeling agents, wherein the second labeling agent indicates the presence of OMP, wherein the presence of the first and second labeling agents on the suspected cell or group of cells indicates that the cell is a GG neuron.

24. A kit for performing the method of claim 19.
